(19) United States
(12) Patent Application Publication
(10) Pub. No.: US 2019/0228367 A1
(43) Pub. Date: Jul. 25, 2019

(54) PROFILE BUILDING USING OCCUPANT STRESS EVALUATION AND PROFILE MATCHING FOR VEHICLE ENVIRONMENT TUNING DURING RIDE SHARING

(71) Applicant: GM Global Technology Operations LLC, Detroit, MI (US)

(72) Inventors: Simone Longo, Turin (IT); Jaime Andres Cespedes Garcia, Turin (IT); Massimiliano Melis, Rivoli (IT); Raffaele Calabrese, Turin (IT)

(73) Assignee: GM Global Technology Operations LLC, Detroit, MI (US)

(21) Appl. No.: 15/878,481

(22) Filed: Jan. 24, 2018

(51) Int. Cl. G06Q 10/06; G06Q 50/30

(52) U.S. Cl. CPC G06Q 10/063112; G16H 20/70; G06Q 50/30

(57) ABSTRACT

A system for generating and matching profiles of people for ride sharing comprises a processor to process physiological data of users collected by sensors in the vehicles, driving data collected by sensors in the vehicles, and lifestyle data of the users. The processor generates user profiles based on the physiological, driving, and lifestyle data. The user profiles include correlations between the data and stress levels of the users during the use of the vehicles. A network interface receives the physiological, driving, and lifestyle data from the vehicles; receives a request from a first user to share a ride in a vehicle; receives information from the processor about a second user having a user profile compatible to the first user; and sends a response to the first user including the information about the second user to allow the first user to share the ride in the vehicle with the second user.

### INTRODUCTION

The information provided in this section is for the purpose of generally presenting the context of the disclosure. Work of the presently named inventors, to the extent it is described in this section, as well as aspects of the description that may not otherwise qualify as prior art at the time of filing, are neither expressly nor impliedly admitted as prior art against the present disclosure.

The present disclosure relates generally to ride sharing and more particularly to generating and matching profiles of people for ride sharing.

Sharing a ride, where two or more persons share a vehicle to go from place A to place B, is becoming increasingly common in major cities where vehicular traffic is problematic. Indeed, some cities provide separate lanes for vehicles with two or more occupants during rush hours to encourage people to share a ride so as to alleviate traffic congestion.

Occupants sharing a ride in a vehicle can have different temperaments and may react differently to stress inducing events such as a traffic jam. These stresses can lead to aggressive behavior sometimes known as road rage, which can further escalate or aggravate stress levels of the occupants. These stresses can occur whether an occupant is driving the vehicle or is simply riding the vehicle. These stresses can also occur when people share a ride in autonomous vehicles, which are on the horizon.

### SUMMARY

A system for generating and matching profiles of people for ride sharing comprises a processor and memory storing instructions for the processor. The processor is configured to execute the instructions to process physiological data of users of vehicles collected by sensors in the vehicles, the physiological data comprising data indicating heart rate, breathing rate, and body movements of the users during use of the vehicles. The processor is configured to execute the instructions to process driving data collected by sensors in the vehicles, the driving data comprising data indicating speed, acceleration, braking, and navigation used during the use of the vehicles. The processor is configured to execute the instructions to process lifestyle data comprising age and gender of the users, infotainment preferences and vehicle environment preferences of the users, and health data indicating health status and exercise habits of the users. The processor is configured to execute the instructions to generate user profiles based on the physiological, driving, and lifestyle data, the user profiles including correlations between the physiological, driving, and lifestyle data and stress levels of the users during the use of the vehicles. The system further comprises a network interface configured to receive the physiological, driving, and lifestyle data from the vehicles; receive a request from a first user to share a ride in a vehicle; receive information from the processor about a second user having a user profile compatible to the first user; and send a response to the first user including the information about the second user to allow the first user to share the ride in the vehicle with the second user.

In other features, the processor is further configured to execute the instructions to select the second user having the user profile compatible to the first user and to send the information about the second user to the network interface.

In other features, the processor is further configured to execute the instructions to generate driving profiles of the users based on the driving data, the driving profiles indicating driving styles of the users; and to select the second user having a driving profile compatible to the first user.

In other features, the processor is further configured to execute the instructions to identify, based on the physiological, driving, and lifestyle data, one or more vehicle parameters that alleviate the stress levels of the users; and to include the one or more vehicle parameters in the user profiles.

In other features, the one or more vehicle parameters comprise settings for infotainment and vehicle environment, navigation, and driving style.

In other features, the infotainment settings comprise settings for one or more of a type of music and a radio station; the vehicle environment settings comprise settings for one or more of interior lighting, ringtones, temperature, fan speed, humidity, and windows/sunroof; the navigation settings comprise alternate routes based on traffic conditions; and the driving style settings comprise one or more of selecting a different route according to speed preferences, using less than preferred speed, and using fewer than preferred lane changes.

In other features, the vehicle in which the first and second users share the ride includes an autonomous vehicle. The processor is further configured to execute the instructions to select, based on the user profiles of the first and second users, a driving configuration comprising one or more of a route for the ride, infotainment and vehicle environment preferences of the first and second users, and driving styles of the first and second users. The network interface is further configured to send the driving configuration to the vehicle.

In other features, one or more subsystems of the vehicle are configured to operate according to the driving configuration when the first and second users share the ride in the vehicle.

In other features, the vehicle in which the first and second users share the ride includes an autonomous vehicle. One or more subsystems of the vehicle are configured to operate according to the user profiles of the first and second users when the first and second users share the ride in the vehicle.

In other features, when the first and second users share the ride in the vehicle, the processor is further configured to execute the instructions to process additional physiological, driving, and lifestyle data received from the vehicle during the ride; and to update the user profiles of the first and second users based on the additional physiological, driving, and lifestyle data.

In other features, when the first and second users share the ride in the vehicle, the processor is further configured to execute the instructions to process additional driving data received from the vehicle during the ride; and to update the driving profiles of the first and second users based on the additional driving data.

In other features, the network interface is configured to receive the request from the first user from a handheld computing device of the first user and to send the information about the second user to the handheld computing device of the first user.

In other features, the network interface is further configured to wirelessly communicate with the vehicles and the first and second users.

In other features, a server includes the processor, the memory, and the network interface; and the server is implemented in a cloud-based computing environment.

In other features, the processor is further configured to execute the instructions to monitor the physiological data of a user for a predetermined period of time during the use of the vehicle; to determine, based on the lifestyle data and the monitored data of the user, values of the physiological data for indicating a baseline stress level and a threshold stress level of the user; and to determine, by monitoring the physiological data of the user, whether a current stress level of the user is greater than or equal to the threshold stress level. When the current stress level of the user is greater than or equal to the threshold stress level, one or more vehicle parameters are changed based on the user profile of the user to reduce the stress level of the user.

In still other features, a method for generating and matching profiles of people for ride sharing, performed by a processor by executing instructions stored in memory, comprises processing, using the processor, physiological data of users of vehicles collected by sensors in the vehicles, the physiological data comprising data indicating heart rate, breathing rate, and body movements of the users during use of the vehicles. The method further comprises processing, using the processor, driving data collected by sensors in the vehicles, the driving data comprising data indicating speed, acceleration, braking, and navigation used during the use of the vehicles. The method further comprises processing, using the processor, lifestyle data comprising age and gender of the users, infotainment preferences and vehicle environment preferences of the users, and health data indicating health status and exercise habits of the users. The method further comprises generating, using the processor, user profiles based on the physiological, driving, and lifestyle data, the user profiles including correlations between the physiological, driving, and lifestyle data and stress levels of the users during the use of the vehicles. The method further comprises receiving a request from a first user to share a ride in a vehicle. The method further comprises identifying a second user having a user profile compatible to the first user. The method further comprises sending information about the second user to allow the first user to share the ride in the vehicle with the second user.

In other features, the method further comprises generating, using the processor, driving profiles of the users based on the driving data, the driving profiles indicating driving styles of the users. The method further comprises selecting the second user having a driving profile compatible to the first user.

In other features, the method further comprises identifying, based on the physiological, driving, and lifestyle data, one or more vehicle parameters that alleviate the stress levels of the users. The method further comprises including the one or more vehicle parameters in the user profiles. The one or more vehicle parameters comprise settings for infotainment and vehicle environment, navigation, and driving style.

In other features, the method further comprises, when the vehicle in which the first and second users share the ride includes an autonomous vehicle, selecting, based on the user profiles of the first and second users, a driving configuration comprising one or more of a route for the ride, infotainment and vehicle environment preferences of the first and second users, and driving styles of the first and second users; sending the driving configuration to the vehicle; and configuring one or more subsystems of the vehicle to operate according to the driving configuration when the first and second users share the ride in the vehicle.

In other features, the method further comprises monitoring the physiological data of a user for a predetermined period of time during the use of the vehicle. The method further comprises determining, based on the lifestyle data and the monitored data of the user, values of the physiological data for indicating a baseline stress level and a threshold stress level of the user. The method further comprises determining, by monitoring the physiological data of the user, whether a current stress level of the user is greater than or equal to the threshold stress level. The method further comprises when the current stress level of the user is greater than or equal to the threshold stress level, changing one or more vehicle parameters based on the user profile of the user to reduce the stress level of the user.

Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 shows a computing device and a plurality of subsystems of a vehicle connected to each other using a Controlled Area Network (CAN) bus in the vehicle;

FIG. 2 shows a simplified example of a distributed network system comprising a plurality of client devices, a plurality of servers, and a plurality of vehicles;

FIG. 3 is a functional block diagram of a simplified example of a client device used in the distributed network system of FIG. 2;

FIG. 4 is a functional block diagram of a simplified example of a server used in the distributed network system of FIG. 2;

FIG. 5 is a flowchart of a method for generating profiles of people for sharing a ride in a vehicle according to the present disclosure;

FIG. 6 is a flowchart of a method for matching profiles of people for sharing a ride in a vehicle according to the present disclosure; and

FIG. 7 is a flowchart of a method for determining baseline stress levels and stress level thresholds of users when generating and updating user profiles of people for ride sharing according to the present disclosure.

In the drawings, reference numbers may be reused to identify similar and/or identical elements.

### DETAILED DESCRIPTION

The present disclosure relates to systems and methods that collect various types of data about users of vehicles and that generate various profiles of the users. For example, vehicles can be equipped with sensors to sense physiological data (e.g., heart rate, breathing rate, etc.) of users while the users ride in a vehicle. Such sensors can be arranged in seats or elsewhere in the vehicle. The vehicles can be further equipped with sensors that sense driving data (e.g., speed, acceleration, braking, and navigation) of the vehicle. The systems and methods can collect the physiological data and the driving data for each user of the vehicle.

The systems and methods can also collect lifestyle data of the user. The lifestyle data may include age and gender of the user. The lifestyle data may further include infotainment preferences (e.g., preferred music, radio stations, etc.) of the user while riding a vehicle. The lifestyle data may additionally include vehicle environment preferences (e.g., preferred temperature, fan speed, interior lighting, etc.) of the user while riding a vehicle. The lifestyle data may also include health data indicating health status and exercise habits of the user.

Based on the physiological, driving, and lifestyle data of users collected from vehicles and users, the systems and methods can generate user profiles of the users. The user profiles can indicate correlations between the physiological, driving, and lifestyle data of users and stress levels of users. For example, a user profile of a user can indicate stress levels of the user in response to different stress stimuli (e.g., driving conditions, behavior of other users in the vehicle, etc.) during a ride in a vehicle. The user profile can also indicate actions that can reduce the stress level of the user during the ride in the vehicle. The user profile can also indicate stress level thresholds or values of the physiological data of the user at which to perform the stress reducing actions during the ride in the vehicle.

Non-limiting examples of the stress reducing actions that may be performed during a ride in a vehicle include playing a particular music or radio station, selecting an alternate route, using or not using cruise control, using or not using an onboard navigation system, setting preferred interior lighting and/or temperature, turning off ringtones or lowering volumes of ringtones of devices, opening or closing windows/sunroof, etc.

Based on the driving data of users collected from the vehicles, the systems and methods can also generate driving profiles of the users. A driving profile of a user can indicate a driving style of the user. For example, the driving profile can indicate whether the user is aggressive (e.g., tendencies for frequent lane changes, speeding, etc.). The driving profile can also indicate whether the user frequently accelerates and brakes or maintains steady speed. The driving profile can provide further indicia of the driving style including preferences regarding using freeways versus surface roads, avoiding route going through crowded areas such as downtowns, distance maintained from other vehicles, accelerating through intersections when the traffic signals are changing, turning on a red signal, etc.

Based on the user profiles and the driving profiles of the users, the systems and methods of the present disclosure can provide suitable matches to a user for sharing a ride. When sharing a ride in autonomous vehicles, only the user profiles and route need to be matched, and a driving configuration can be downloaded to the vehicle. When sharing a ride in non-autonomous vehicles, the user profiles, the driving profiles, and the route need to be matched. These and other aspects of the present disclosure are described below in detail.

For example, when a request to share a ride with others is received from a first user, the systems and methods can compare the user profile of the first user to user profiles of other users and identify users having user profiles that are compatible with the user profile of the first user. For example, a second user may have a user profile similar to the user profile of the first user (e.g., both users may have similar stress profiles and similar preferences regarding actions that can alleviate stress). Alternatively, the second user may have a user profile that is complementary to the user profile of the first user (e.g., the second user may be less susceptible to stress than the first user and therefore may have a calming impact on the first user if the two users ride together). The systems and methods can send the information about the second user with a similar user profile to the first user. The first user can then share a ride with the second user.

In addition to matching the user profiles, if sharing a ride in a non-autonomous vehicle, the systems and methods can additionally compare the driving profile of the first user to driving profiles of other users and identify users having driving profiles that are also compatible with the driving profile of the first user. For example, the second user having a similar user profile to the first user may also have a driving profile that is similar to the driving profile of the first user (e.g., both users may have similar driving styles). The systems and methods can send information about the second user with a similar driving profile and a similar user profile to the first user. The first user can then share a ride with the second user.

The systems and methods can be implemented using one or more servers as explained below in detail. For example, the systems and methods can be implemented in a cloud computing environment. The various data can be collected from the users and vehicles via distributed communication systems such as the Internet and cellular and other wireless networks. The users can communicate with the servers using computing devices (e.g., smartphones). The vehicles can communicate with the servers using onboard computing devices. These and other features of the systems and methods of the present disclosure are now described in further detail.

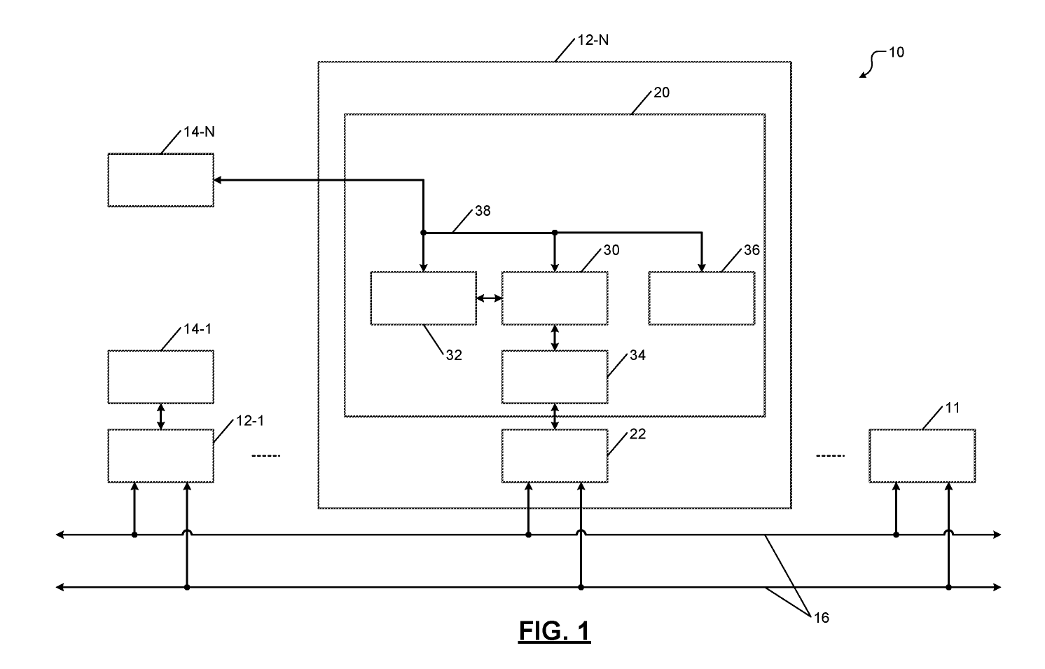

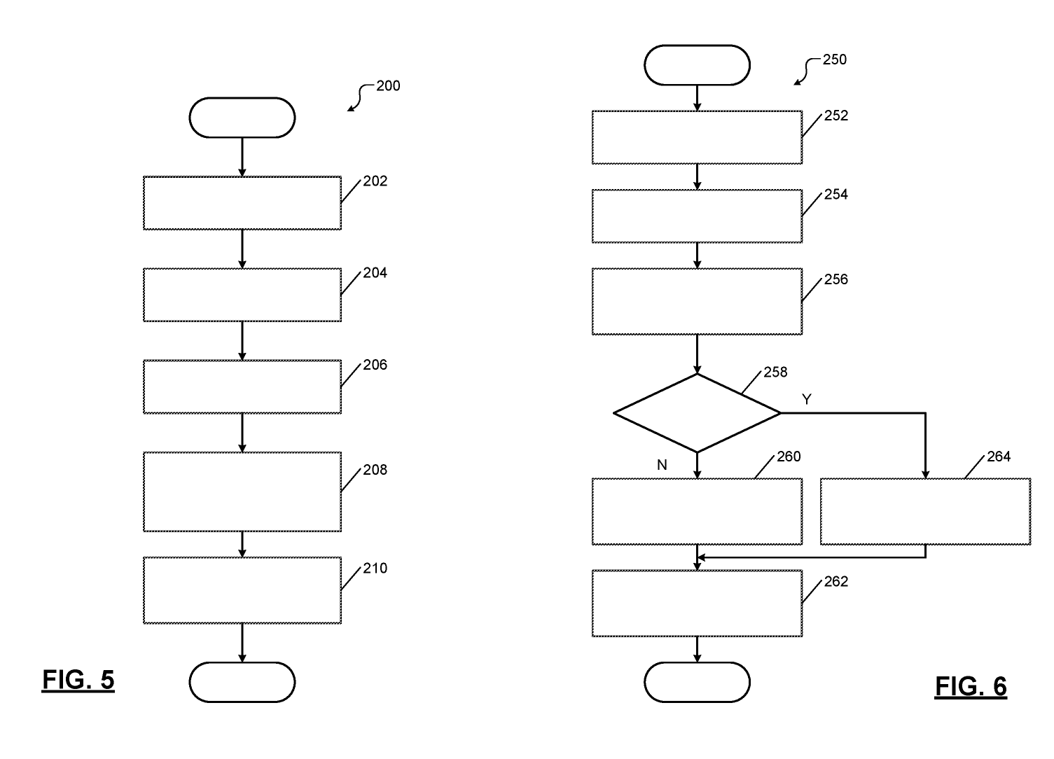

The present disclosure is organized as follows. FIG. 1 shows a computing device and various subsystems of a vehicle connected to each other using a Controlled Area Network (CAN) bus. FIGS. 2-4 show simplistic examples of a distributed computing environment in which the systems and methods of the present disclosure can be implemented. FIGS. 5-7 show the systems and methods of the present disclosure in detail.

Throughout the present disclosure, references to terms such as servers, client devices, applications, and so on are for illustrative purposes only. The terms servers and client devices are to be understood broadly as representing computing devices comprising one or more processors and memory configured to execute machine readable instructions. The terms applications and computer programs are to be understood broadly as representing machine readable instructions executable by the computing devices.

Automotive electronic control systems are typically implemented as Electronic Control Units (ECU's) that are connected to each other by a Controller Area Network (CAN) bus. Each ECU controls a specific subsystem (e.g., engine, transmission, heating and cooling, infotainment, navigation, and so on) of the vehicle. Each ECU includes a microcontroller, a CAN controller, and a transceiver. In each ECU, the microcontroller includes a processor, memory, and other circuits to control the specific subsystem. Each ECU can communicate with other ECU's via the CAN bus through the CAN controller and the transceiver.

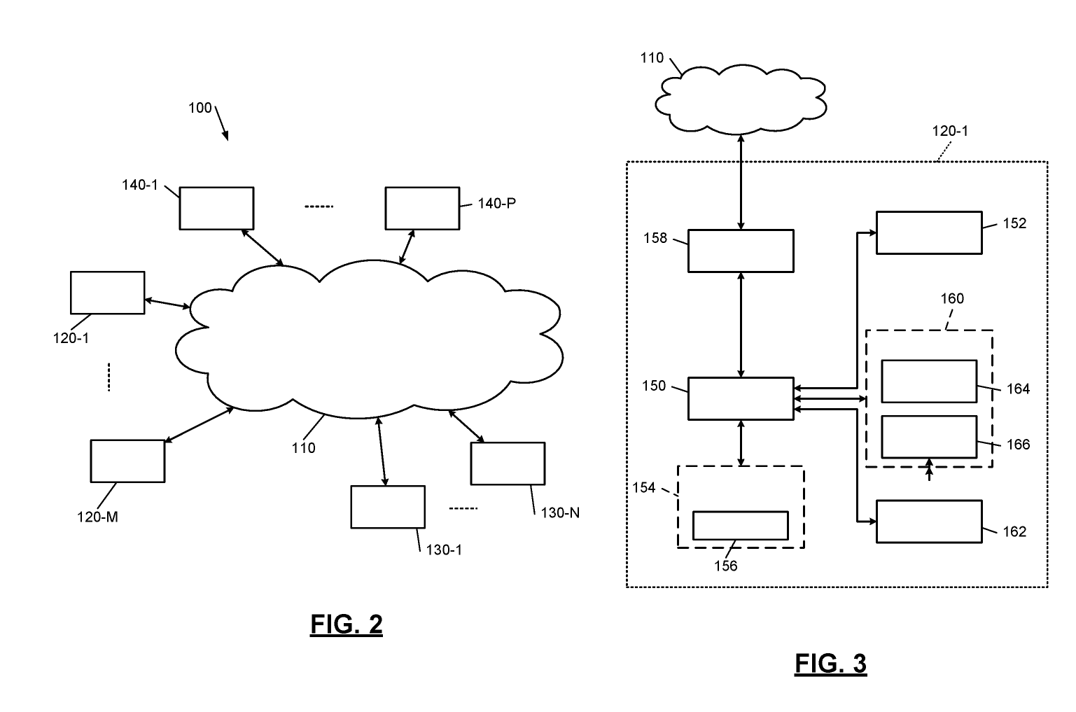

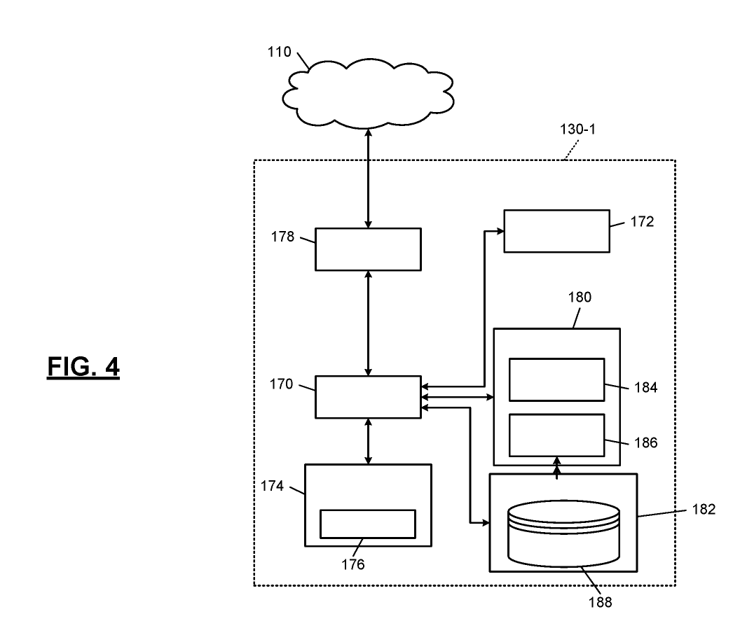

FIG. 1 shows an example of a vehicle 10 comprising a computing device 11 and a plurality of ECU's connected to each other by a CAN bus. The computing device 11 is similar to a client device 120 shown in FIG. 3 and is therefore not described here. In addition to including the components of the client device 120 shown in FIG. 3, the computing device 11 includes one or more components of an ECU 12 described below. Accordingly, the computing device 11 can communicate with the CAN bus and can interface (i.e., exchange data) with the ECU's 12 via the CAN bus.

The plurality of ECU's includes ECU-1 12-1, ECU-2 12-2, . . . , and ECU-N 12-N (collectively, ECU's 12), where N is an integer greater than one. Hereinafter, ECU 12 refers to any of the plurality of ECU's 12. While FIG. 1 shows a detailed functional block diagram of only the ECU-N 12-N, it will be understood that other ECUs 12 can have structure similar to the ECU-N 12-N. Each ECU 12 or any portion thereof may be implemented as one or more modules.

Each ECU 12 controls a respective subsystem of the vehicle 10. For example, the ECU-1 12-1 controls a subsystem 14-1, the ECU-2 12-2 controls a subsystem 14-2, . . . , and the ECU-N 12-N controls a subsystem 14-N. Collectively the subsystems 14-1, 14-2, . . . , and 14-N are referred to as subsystems 14. Non-limiting examples of the subsystems 14 include an infotainment subsystem, a navigation subsystem, a physiological data acquisition subsystem, a driving data acquisition subsystem, an engine control subsystem, a transmission control subsystem, a brake control subsystem, an exhaust control subsystem, a traction control subsystem, a suspension control subsystem, a climate control subsystem, a safety subsystem, and so on.

Each subsystem 14 may include one or more sensors to sense data from one or more components of the subsystem 14. For example, the physiological data acquisition subsystem may include biometric or biological sensors and cameras to collect physiological data from occupants of the vehicle 10; the driving data acquisition subsystem may include sensors to collect driving data such as speed, acceleration, braking, and navigation data of the vehicle 10; the safety subsystem may include cameras; and so on. Each subsystem 14 may include one or more actuators to actuate one or more components of the subsystem 14.

An ECU 12 may receive data from one or more sensors of a corresponding subsystem 14. Depending on the type of ECU, the ECU 12 may also receive one or more inputs from an occupant of the vehicle 10. The ECU 12 may control one or more actuators of the corresponding subsystem 14 based on the data received from the one or more sensors and/or the one or more inputs from an occupant of the vehicle 10.

The ECUs 12 are connected to a CAN bus 16. The ECUs 12 can communicate with each other and with the computing device 11 via the CAN bus 16. The ECUs 12 can communicate with other devices connected to the CAN bus 16 (e.g., test equipment, a communication gateway, etc.). Each ECU 12 includes a microcontroller 20 and a CAN transceiver 22. The microcontroller 20 communicates with the subsystem 14 controlled by the ECU 12. The CAN transceiver 22 communicates with the CAN bus 16.

The microcontroller 20 includes a processor 30, a memory 32, a CAN controller 34, and a power supply 36. The memory 32 includes volatile memory (RAM) and may additionally include nonvolatile memory (e.g., flash memory) and/or other type of data storage device(s). The processor 30 and the memory 32 communicate with each other via a bus 38. The processor 30 executes code stored in the memory 32 to control the subsystem 14. The power supply 36 supplies power to all of the components of the microcontroller 20 and the ECU 12. The CAN controller 34 communicates with the CAN transceiver 22.

The computing device 11 can collect the physiological and driving data from the ECU's 12 controlling the respective subsystems 14. The computing device 11 can send the collected physiological and driving data to a remote server (e.g., a server 130 shown in FIGS. 2-4) for analysis and profile building as described below. The computing device 11 can receive data from a remote server (e.g., a server 130 shown in FIGS. 2-4). For example, the computing device 11 can receive autonomous vehicle configuration and profiles of users riding the vehicle 10. The computing device 11 can send the data (e.g., the autonomous vehicle configuration and profiles of users) received from a remote server (e.g., a server 130 shown in FIGS. 2-4) to the ECU's 12 controlling the respective subsystems 14. Accordingly, the computing device 11 can configure one or more subsystems 14 of the vehicle 10 to operate according to the autonomous vehicle configuration (when the vehicle 10 includes an autonomous vehicle) and according to the profiles of users riding the vehicle 10.

In addition, the computing device 11 can store user profiles received from a remote server (e.g., a server 130 shown in FIGS. 2-4). One or more ECU's 12 (e.g., the infotainment ECU, the navigation ECU, and so on) can control respective subsystems 14 according to the user profiles. For example, when users share a ride in the vehicle 10, one or more vehicle parameters (e.g., music, radio station, interior lighting, temperature, fan speed, navigation, speed, acceleration, braking, etc.) are controlled by respective subsystems 14 according to the user profiles of the users stored in the computing device 11. The computing device 11 interacts with the subsystems 14 via the respective ECU's 12 to coordinate the control of one or more vehicle parameters according to the user profiles of the users stored in the computing device 11. These aspects of the present disclosure are described below in further detail.

FIG. 2 shows a simplified example of a distributed network system 100. The distributed network system 100 includes a network 110 (e.g., a distributed communication system). The distributed network system 100 includes one or more client devices 120-1, 120-2, . . . , and 120-M (collectively client devices 120); one or more servers 130-1, 130-2, . . . , and 130-N (collectively servers 130); and one or more vehicles 140-1, 140-2, . . . , and 140-P (collectively vehicles 140), where M is an integer greater than 1, and where N and P are integers greater than or equal to 1.

The network 110 may include a local area network (LAN), a wide area network (WAN) such as the Internet, a cellular network, or other type of network (collectively shown as the network 110). The client devices 120 may include computing devices (e.g., smartphones) and may communicate with the servers 130 via the network 110. The client devices 120 and the servers 130 may connect to the network 110 using wireless and/or wired connections to the network 110. Throughout the present disclosure, references to the client devices 120 are to be understood as references to respective users of the client devices 120.

Each vehicle 140 comprises the computing device 11 (that is similar to the client device 120), the ECU's 12, and the subsystems 14 shown in FIG. 1. Throughout the present disclosure, communications with and by the vehicles 140 are to be understood as communications with the computing devices 11 in the vehicles 140. The computing device 11 in each vehicle 140 may execute applications that communicate with various sensors in the vehicle 140 that sense the physiological data and the driving data of users riding the vehicle 140. The computing device 11 in each vehicle 140 may also execute applications that communicate with various subsystems 14 of the vehicle 140. Non-limiting examples of the various subsystems of the vehicle 140 includes infotainment system, navigation system, HVAC system, and other control systems that control various operations of the vehicle 140. The computing devices 11 in the vehicles 140 may communicate with the servers 130 via the network 110. The vehicles 140 (i.e., the computing devices of the vehicles 140) may communicate the network 110 using wireless connections to the network 110.

The servers 130 may provide multiple services to the client devices 120 and to the computing devices 11 in the vehicles 140 (i.e., to the vehicles 140). For example, the servers 130 may execute a plurality of software applications. The servers 130 may host multiple databases that are utilized by the plurality of software applications and that are used by the client devices 120 and the vehicles 140. In addition, the servers 130, the client devices 120, and the computing devices in the vehicles 140 may execute applications that implement at least some portions of the methods described below with reference to FIGS. 5-7.

FIG. 3 shows a simplified example of the client devices 120 (e.g., the client device 120-1). The client device 120-1 may typically include a central processing unit (CPU) or processor 150, one or more input devices 152 (e.g., a keypad, touchpad, mouse, and so on), a display subsystem 154 including a display 156, a network interface 158, a memory 160, and a bulk storage 162.

The network interface 158 connects the client device 120-1 to the distributed network system 100 via the network 110. For example, the network interface 158 may include a wired interface (e.g., an Ethernet interface) and/or a wireless interface (e.g., a Wi-Fi, Bluetooth, near field communication (NFC), cellular, or other wireless interface). The memory 160 may include volatile or nonvolatile memory, cache, or other type of memory. The bulk storage 162 may include flash memory, a hard disk drive (HDD), or other bulk storage device.

The processor 150 of the client device 120-1 may execute an operating system (OS) 164 and one or more client applications 166. The client applications 166 may include an application to connect the client device 120-1 to the servers 130 via the network 110. The client device 120-1 may access one or more applications executed by the servers 130 via the network 110. The client applications 166 may also include an application that implements one or more portions of the methods described below with reference to FIGS. 5-7.

For example, a client application 166 on a smartphone of a user (i.e., on a client device 120) may send a request for sharing a ride to one of the servers 130 and may receive information from one of the servers 130 regarding one or more users suitable for sharing the ride. A client application 166 on a computing device 11 of a vehicle 140 may send the physiological data and the driving data to one of the servers 130. In case of autonomous vehicles, the client application 166 on a computing device 11 of a vehicle 140 may receive a driving configuration from one of the servers 130 when two or more users decide to share a ride in the vehicle 140.

FIG. 4 shows a simplified example of the servers 130 (e.g., the server 130-1). The server 130-1 typically includes one or more CPUs or processors 170, one or more input devices 172 (e.g., a keypad, touchpad, mouse, and so on), a display subsystem 174 including a display 172, a network interface 178, a memory 180, and a bulk storage 182.

The network interface 178 connects the server 130-1 to the distributed network system 100 via the network 110. For example, the network interface 178 may include a wired interface (e.g., an Ethernet interface) and/or a wireless interface (e.g., a Wi-Fi, Bluetooth, near field communication (NFC), cellular, or other wireless interface). The memory 180 may include volatile or nonvolatile memory, cache, or other type of memory. The bulk storage 182 may include flash memory, one or more hard disk drives (HDDs), or other bulk storage device.

The processor 170 of the server 130-1 may execute an operating system (OS) 184 and one or more server applications 186. The server applications 186 may include an application that implements the methods described below with reference to FIGS. 5-7. The bulk storage 182 may store one or more databases 188 that store data structures used by the server applications 186 to perform respective functions.

In FIGS. 5-7, various methods for generating and matching profiles of people for ride sharing are shown. These methods are implemented by the applications executed by the servers 130, the vehicles 140 (i.e., the computing devices 11 in the vehicles 140), and the client devices 120. In the following description, the term control represents code or instructions executed by one or more components of the servers 130, the vehicles 140 (i.e., the computing devices 11 in the vehicles 140), and the client devices 120 shown in FIGS. 1-3. The term control refers to one or more of the server applications 186, client applications 166, and the applications executed by the computing devices 11 in the vehicles 140 that are described above with reference to FIGS. 2-4.

FIG. 5 shows a method 200 for generating profiles of people for sharing a ride in a vehicle according to the present disclosure. The method 200 can be executed on one or more of the servers 130 (e.g., by the applications 186) and one or more of the vehicles 140 (e.g., by the applications executed on the computing devices 11 in one or more of the vehicles 140). For example, sensing the various types of data as described below can be performed by the applications executed on the computing devices 11 in one or more of the vehicles 140. The processing of the sensed data and the generation of the profiles as described below can be performed by the applications 186 executed on one or more of the servers 130.

At 202, control (e.g., a server 130) receives the physiological data of the users from the vehicles 140 (i.e., from the computing devices 11 in the vehicles 140) through the network 110 (e.g., via the network interface 178). The physiological data comprises data indicating heart rate, breathing rate, and body movements of the users during the use of the vehicles 140. Non-limiting examples of body movements can include fidgeting, eye gaze, percentage of eye enclosure, eye blinking, head tilt, facial expressions, facial color, perspiration, and so on. The physiological data is sensed by various sensors including one or more cameras located throughout the vehicles 140. The physiological data is wirelessly transmitted from the vehicles 140 (i.e., from the computing devices 11 in the vehicles 140) to the servers 130 through the network 110.

At 204, control (e.g., a server 130) receives the driving data of the users from the vehicles 140 (i.e., from the computing devices 11 in the vehicles 140) through the network 110 (e.g., via the network interface 178). The driving data comprises data indicating speed, acceleration, braking, and navigation used during the use of the vehicles 140. The driving data is sensed by various sensors including one or more cameras located throughout the vehicles 140. The driving data is wirelessly transmitted from the vehicles 140 (i.e., from the computing devices 11 in the vehicles 140) to the servers 130 through the network 110.

At 206, control (e.g., a server 130) receives the lifestyle data of the users from the client devices 120 of the users through the network 110 (e.g., via the network interface 178). The lifestyle data comprises data indicating age and gender of the users, infotainment preferences and vehicle environment preferences of the users, and health data indicating health status and exercise habits of the users. The lifestyle data is wirelessly transmitted from the client devices 120 of the users to the servers 130 through the network 110.

Non-limiting examples of infotainment preferences include preferred music and/or radio stations. Non-limiting examples of vehicle environment preferences include preferred interior lighting in the vehicles 140, ring tone volume of devices being used in the vehicles 140; temperature, fan speed, and humidity in the vehicles 140; whether windows/sunroof of the vehicles 140 should be open or shut; etc.

Non-limiting examples of health data include any heart and/or lung conditions that could affect baseline values of the corresponding physiological data (e.g., heart rate, breathing rate, and so on). Exercise habits can affect baseline values of the corresponding physiological data (e.g., heart rate, breathing rate, and so on). The exercise habits can also reduce stress levels of the users, which can change their stress level thresholds that initiate or trigger countermeasures or actions needed to alleviate the stress levels.

At 208, control (e.g., a processor 170 of a server 130) processes the physiological, driving, and lifestyle data of the users and generates user profiles based on the analysis of the physiological, driving, and lifestyle data of the users. The user profiles include correlations between the physiological, driving, and lifestyle data of the users and stress levels of the users during the use of the vehicles 140. The user profiles include a correspondence between values of the physiological data and stress levels of the users in response to different stress stimuli. The user profiles include stress level thresholds that can be used to trigger or initiate actions that reduce the stress levels. Control also continues to update these user profiles during the use of the vehicles 140 (i.e., during ride sharing) to refine the user profiles through a continued learning process.

At 210, control (e.g., a processor 170 of a server 130) processes the driving data of the users and generates driving profiles based on the analysis of the driving data of the users. The driving profiles indicate driving styles and habits of the users. Control also continues to update the driving profiles during the use of the vehicles 140 (i.e., during ride sharing) to refine the driving profiles through a continued learning process. The user profiles and the driving profiles can be stored in one or more databases (e.g., the databases 188) of the servers 170.

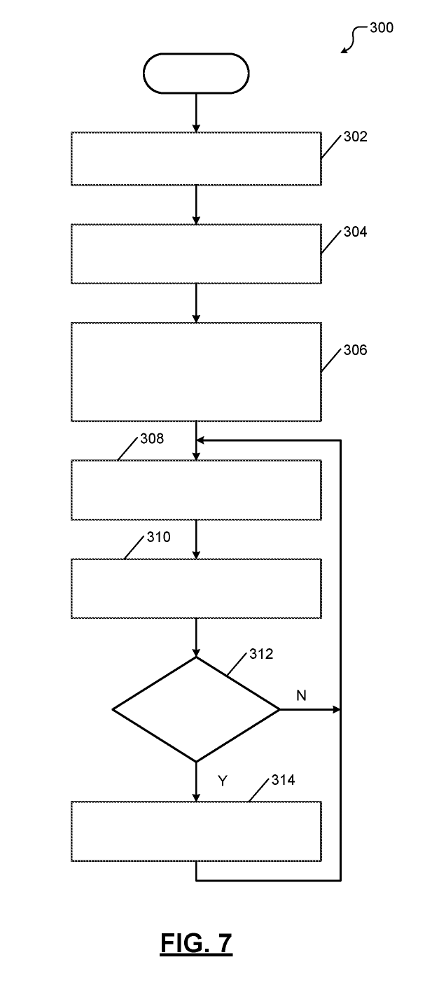

FIG. 6 shows a method 250 for matching profiles of people for sharing a ride in a vehicle according to the present disclosure. The method 250 can be executed on one or more of the servers 130 (e.g., by the applications 186) and one or more of the client devices 120 of the users (e.g., by the applications 166). For example, the applications 166 executed on one or more of the client devices 120 of the users can send ride share requests and can receive information about users with whom a ride can be shared. The applications 186 executed on one or more of the servers 130 can receive ride share requests, identify matching profiles, send information about users with matching profiles, and download a driving configuration to one of the vehicles 140 in which the identified users can share the ride.

At 252, control (e.g., a server 130) receives a request to share a ride from a first user (e.g., from one of the client devices 120) through the network 110 (e.g., via the network interface 178). The request may include a start time, a start location, and a destination for the ride. At 254, control (e.g., a processor 170 of a server 130) identifies one or more users with a matching rout (i.e., users interested in traveling the same route as the first user). At 256, control (e.g., a processor 170 of a server 130) identifies users with user profiles (stored in one or more databases (e.g., the databases 188) of the servers 170) compatible with the user profile of the first user.

At 258, control determines whether the vehicle 140 being shared is an autonomous vehicle. At 260, if the vehicle 140 being shared is not an autonomous vehicle, control (e.g., a processor 170 of a server 130) identifies users with driving profiles (stored in one or more databases (e.g., the databases 188) of the servers 170) compatible with the driving profile of the first user. That is, from the users that are already identified at 256 as having user profiles compatible with the user profile of the first user, control further identifies one or more of these users that additionally have driving profiles compatible with the driving profile of the first user. Accordingly, control identifies one or more users having user profiles and driving profiles that are compatible with the user profile and the driving profile of the first user.

At 262, if the vehicle 140 being shared is an autonomous vehicle, control (e.g., a processor 170 of a server 130) determines a driving configuration to be sent to the autonomous vehicle. Control determines the driving configuration based on the route and the user profiles of the users identified as having compatible user profiles to share the ride in the autonomous vehicle. The driving configuration comprises the route for the ride and the infotainment and vehicle environment preferences and driving styles of the users identified as having compatible user profiles to share the ride in the autonomous vehicle.

At 264, control (e.g., a server 130) sends information about the users having compatible profiles to the first user (e.g., to the client device 120 that requested ride sharing at 252) through the network 110 (e.g., via the network interface 178). If the vehicle 140 being shared is not an autonomous vehicle, control sends information about the users having compatible user profiles and compatible driving profiles to the first user. If the vehicle 140 being shared is an autonomous vehicle, control sends information about the users having compatible user profiles to the first user.

Based on the information received, the first user can communicate with the users (e.g., client devices 120 of the users) identified as having user profiles and optionally also driving profiles compatible with the first user, and the first user can share the ride with these users in one of the vehicles 140. During the shared ride, one or more vehicle parameters can be changed depending on the stress levels of the users according to the user profiles and optionally also according to the driving profiles of the users sharing the ride.

The vehicle parameters comprise settings for infotainment and vehicle environment, navigation, and driving style to be used during the ride. Non-limiting examples of the infotainment settings include choice of music and or radio station in the vehicle 140 being shared by the users. Non-limiting examples of the vehicle environment settings include settings for interior lighting, ring tones, temperature, fan speed, humidity, and windows/sunroof in the vehicle 140 being shared by the users. Non-limiting examples of the navigation settings comprise settings for selecting alternate routes based on traffic conditions during the ride. Non-limiting examples of the driving style settings include using less than preferred speed and/or acceleration for the vehicle 140, and fewer than preferred lane changes during the ride.

FIG. 7 shows a method 300 for determining baseline stress levels and stress level thresholds of users when generating and updating user profiles of people for ride sharing according to the present disclosure. The method 300 also determines when to change vehicle parameters based on the user profiles. The method 300 can be executed on one or more of the servers 130 (e.g., by the applications 186), one or more of the client devices 120 of the users (e.g., by the applications 166), and one or more of the vehicles 140 (e.g., by the applications executed by the computing devices 11 in the vehicles 140).

For example, users can send their lifestyle data using the applications 166 executed on one or more of the client devices 120 of the users. The vehicles 140 can sense the physiological data of the users during a ride and send the sensed physiological data to one or more of the servers 130 using the applications executed by the computing devices 11 in the vehicles 140. The applications 186 executed on one or more of the servers 130 can monitor (i.e., receive physiological data), analyze the physiological data, determine baseline stress levels and threshold stress levels of the users, and determine if a current stress level exceeds a threshold stress level. The applications 186 executed on one or more of the servers 130 and the applications executed by the computing devices 11 in the vehicles 140 can initiate changes in one or more vehicle parameters to reduce the current stress level according to the user profile of the user.

At 302, control receives the lifestyle data of a user. For example, the user may provide the lifestyle data including age, gender, infotainment and vehicle environment preferences, and health data indicating health status and exercise habits of the user.

At 304, control monitors the physiological data of the user for a predetermined period of time during initial use of the vehicle. Examples of the physiological data are already provided above and are therefore not repeated for brevity. Control may select one or more types of the physiological data (e.g., heart rate and breathing rate) instead of considering all types of the physiological data.

At 306, based on the lifestyle data and the monitored physiological data, control determines values of the physiological data as indicating a baseline stress level of the user. Control also determines values of the physiological data as indicating a threshold stress level for the user based on the lifestyle data and the monitored physiological data.

At 308, after determining the baseline stress level and the threshold stress level for the user, control continues to monitor the physiological data of the user during the use of the vehicle. At 310, control determines a current stress level of the user based on the current physiological data of the user. At 312, control determines whether the current stress level of the user is greater than or equal to the threshold stress level of the user. Control returns to 308 if the current stress level of the user is less than the threshold stress level of the user. At 314, if the current stress level of the user is greater than or equal to the threshold stress level of the user, control changes one or more vehicle parameters according to the user profile of the user to reduce the current stress level of the user, and control returns to 308. A similar method may be used during generation and updating of the user profiles.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

None of the elements recited in the claims are intended to be a means-plus-function element within the meaning of 35 U.S.C. § 112(f) unless an element is expressly recited using the phrase “means for,” or in the case of a method claim using the phrases “operation for” or “step for.”

What is claimed is:

1. A system for generating and matching profiles of people for ride sharing, the system comprising:
a processor and memory storing instructions for the processor, the processor configured to execute the instructions to:
process physiological data of users of vehicles collected by sensors in the vehicles, the physiological data comprising data indicating heart rate, breathing rate, and body movements of the users during use of the vehicles;
process driving data collected by sensors in the vehicles, the driving data comprising data indicating speed, acceleration, braking, and navigation used during the use of the vehicles;
process lifestyle data comprising age and gender of the users, infotainment preferences and vehicle environment preferences of the users, and health data indicating health status and exercise habits of the users; and
generate user profiles based on the physiological, driving, and lifestyle data, the user profiles including correlations between the physiological, driving, and lifestyle data and stress levels of the users during the use of the vehicles; and
a network interface configured to:
receive the physiological, driving, and lifestyle data from the vehicles;
receive a request from a first user to share a ride in a vehicle;
receive information from the processor about a second user having a user profile compatible to the first user; and
send a response to the first user including the information about the second user to allow the first user to share the ride in the vehicle with the second user.

2. The system of claim 1 wherein the processor is further configured to execute the instructions to select the second user having the user profile compatible to the first user and to send the information about the second user to the network interface.

3. The system of claim 1 wherein the processor is further configured to execute the instructions to:
generate driving profiles of the users based on the driving data, the driving profiles indicating driving styles of the users; and
select the second user having a driving profile compatible to the first user.

4. The system of claim 1 wherein the processor is further configured to execute the instructions to:
identify, based on the physiological, driving, and lifestyle data, one or more vehicle parameters that alleviate the stress levels of the users; and
include the one or more vehicle parameters in the user profiles.

5. The system of claim 4 wherein the one or more vehicle parameters comprise settings for infotainment and vehicle environment, navigation, and driving style.

6. The system of claim 5 wherein:
the infotainment settings comprise settings for one or more of a type of music and a radio station;
the vehicle environment settings comprise settings for one or more of interior lighting, ringtones, temperature, fan speed, humidity, and windows/sunroof;
the navigation settings comprise alternate routes based on traffic conditions; and
the driving style settings comprise one or more of selecting a different route according to speed preferences, using less than preferred speed, and using fewer than preferred lane changes.

7. The system of claim 1 wherein:
the vehicle in which the first and second users share the ride includes an autonomous vehicle;
the processor is further configured to execute the instructions to select, based on the user profiles of the first and second users, a driving configuration comprising one or more of a route for the ride, infotainment and vehicle environment preferences of the first and second users, and driving styles of the first and second users; and
the network interface is further configured to send the driving configuration to the vehicle.

8. The system of claim 7 wherein one or more subsystems of the vehicle are configured to operate according to the driving configuration when the first and second users share the ride in the vehicle.

9. The system of claim 1 wherein:
the vehicle in which the first and second users share the ride includes an autonomous vehicle; and
one or more subsystems of the vehicle are configured to operate according to the user profiles of the first and second users when the first and second users share the ride in the vehicle.

10. The system of claim 1 wherein when the first and second users share the ride in the vehicle, the processor is further configured to execute the instructions to:
process additional physiological, driving, and lifestyle data received from the vehicle during the ride; and
update the user profiles of the first and second users based on the additional physiological, driving, and lifestyle data.

11. The system of claim 3 wherein when the first and second users share the ride in the vehicle, the processor is further configured to execute the instructions to:
process additional driving data received from the vehicle during the ride; and
update the driving profiles of the first and second users based on the additional driving data.

12. The system of claim 1 wherein the network interface is configured to:
receive the request from the first user from a handheld computing device of the first user; and
send the information about the second user to the handheld computing device of the first user.

13. The system of claim 1 wherein the network interface is further configured to wirelessly communicate with the vehicles and the first and second users.

14. The system of claim 1 wherein a server includes the processor, the memory, and the network interface; and wherein the server is implemented in a cloud-based computing environment.

15. The system of claim 1 wherein the processor is further configured to execute the instructions to:
monitor the physiological data of a user for a predetermined period of time during the use of the vehicle;
determine, based on the lifestyle data and the monitored data of the user, values of the physiological data for indicating a baseline stress level and a threshold stress level of the user; and
determine, by monitoring the physiological data of the user, whether a current stress level of the user is greater than or equal to the threshold stress level, wherein when the current stress level of the user is greater than or equal to the threshold stress level, one or more vehicle parameters are changed based on the user profile of the user to reduce the stress level of the user.

16. A method for generating and matching profiles of people for ride sharing, the method performed by a processor by executing instructions stored in memory, the method comprising:
processing, using the processor, physiological data of users of vehicles collected by sensors in the vehicles, the physiological data comprising data indicating heart rate, breathing rate, and body movements of the users during use of the vehicles;
processing, using the processor, driving data collected by sensors in the vehicles, the driving data comprising data indicating speed, acceleration, braking, and navigation used during the use of the vehicles;
processing, using the processor, lifestyle data comprising age and gender of the users, infotainment preferences and vehicle environment preferences of the users, and health data indicating health status and exercise habits of the users; and
generating, using the processor, user profiles based on the physiological, driving, and lifestyle data, the user profiles including correlations between the physiological, driving, and lifestyle data and stress levels of the users during the use of the vehicles;
receiving a request from a first user to share a ride in a vehicle;
identifying a second user having a user profile compatible to the first user; and
sending information about the second user to allow the first user to share the ride in the vehicle with the second user.

17. The method of claim 16 further comprising:
generating, using the processor, driving profiles of the users based on the driving data, the driving profiles indicating driving styles of the users; and
selecting the second user having a driving profile compatible to the first user.

18. The method of claim 16 further comprising:
identifying, based on the physiological, driving, and lifestyle data, one or more vehicle parameters that alleviate the stress levels of the users; and
including the one or more vehicle parameters in the user profiles,
wherein the one or more vehicle parameters comprise settings for infotainment and vehicle environment, navigation, and driving style.

19. The method of claim 16 further comprising, when the vehicle in which the first and second users share the ride includes an autonomous vehicle:
selecting, based on the user profiles of the first and second users, a driving configuration comprising one or more of a route for the ride, infotainment and vehicle environment preferences of the first and second users, and driving styles of the first and second users;
sending the driving configuration to the vehicle; and
configuring one or more subsystems of the vehicle to operate according to the driving configuration when the first and second users share the ride in the vehicle.

20. The method of claim 16 further comprising:
monitoring the physiological data of a user for a predetermined period of time during the use of the vehicle;
determining, based on the lifestyle data and the monitored data of the user, values of the physiological data for indicating a baseline stress level and a threshold stress level of the user;
determining, by monitoring the physiological data of the user, whether a current stress level of the user is greater than or equal to the threshold stress level; and
when the current stress level of the user is greater than or equal to the threshold stress level, changing one or more vehicle parameters based on the user profile of the user to reduce the stress level of the user.
